(19) United States
(12) Patent Application Publication
(10) Pub. No.: US 2002/0193363 A1
(43) Pub. Date: Dec. 19, 2002

(54) Use of nitric oxide scavengers to modulate inflammation and matrix metalloproteinase activity

(76) Inventors: Gary J. Bridger, Bellingham, WA (US); Simon P. Fricker, Langley (CA); Michael J. Abrams, Glenmore, PA (US); Irvin Mayers, Edmonton (CA)

Correspondence Address: MORRISON & FOERSTER LLP, 3811 VALLEY CENTRE DRIVE, SUITE 500, SAN DIEGO, CA, 92130-2332, US

(21) Appl. No.: 10/017,764

(22) Filed: Dec. 7, 2001

Related U.S. Application Data
(63) Continuation-in-part of application No. 09/802,523, filed on Mar. 9, 2001, now Pat. No. 6,417,182.
Application No. 09/802,523 is a continuation of application No. 09/175,028, filed on Oct. 19, 1998, now Pat. No. 6,284,752.
Application No. 09/175,028 is a continuation of application No. 08/602,814, filed on Feb. 26, 1996, now Pat. No. 5,824,673.

(51) Int. Cl. A61K 31/555; A61K 31/28

(52) U.S. Cl. 514/185; 514/492

(57) ABSTRACT

The present invention relates to modulating inflammation, or improving hemodynamic function, associated with trauma, such as that caused by surgery, through the administration of metal complexes, such as ruthenium nitric oxide scavengers. Further, the present invention relates to modulating matrix metalloproteinase activity also by administering such metal complexes. Modulation of the trauma or the metalloproteinase activity reduces tissue damage or causes a protective effect against organ damage.

### CROSS-REFERENCE TO RELATED APPLICATIONS

This application is continuation-in-part application of pending U.S. patent application Ser. No. 09/802,523, which is a continuation of U.S. patent application Ser. No. 09/175,028, now U.S. Pat. No. 6,284,752, which is a continuation of U.S. patent application Ser. No. 08/602,814, now U.S. Pat. No. 5,824,673.

### TECHNICAL FIELD

The present invention relates to modulating inflammation, or improving hemodynamic function, associated with trauma caused by surgery, through the administration of metal complexes, such as ruthenium nitric oxide scavengers. Further, the present invention relates to modulating matrix metalloproteinase activity also by administering such metal complexes. Modulation of the surgical trauma or the metalloproteinase activity reduces tissue damage or causes a protective effect against organ damage.

### BACKGROUND OF THE INVENTION

Coronary artery bypass grafting (CABG) is an invasive surgical procedure undertaken to redirect or “bypass” blood around a discrete point(s) within a clogged artery supplying the heart. These arteries can become clogged through the build-up of fat, cholesterol, or other substances, leading to a narrowing or blockage referred to as an atherosclerotic plaque. CABG is a frequently performed operation undertaken throughout the world, with an estimated 800,000 cardiac surgeries worldwide reported in 1997 (American College of Cardiology).

Without CABG surgery, atherosclerosis can impede the flow of blood through the vessels supplying the heart, resulting in myocardial infarcts or heart attacks. During the operation, healthy segments of blood vessels (both arteries and veins) from other regions of the body, for example the saphenous veins from the leg, are used in this operation. For a majority of the CABG surgery performed, a heart-lung machine is used, causing blood to be exposed to an extra-corporeal (outside of the body) circuit. The surgical procedure involves grafting the healthy blood vessels onto the coronary vessel before the point of damage and re-directing the flow of blood to other regions of the heart and circulatory system.

Due to the invasiveness of the pulmonary bypass procedure, CABG is associated with inducing systemic inflammation, by releasing a host of inflammatory mediators from macrophages, including interleukin-6 (IL-6) and tumor necrosis factor (TNF) (Lahat et al. Clin. Exp. Immunol. 89:255-260, 1992), where both IL-6 and TNF in turn may induce the inducible form of nitric oxide synthase (iNOS) activity, one of a family of enzymes responsible for the production of nitric oxide (Cunha et al. Immunology. 81:211-215, 1994); and matrix metalloproteinases (MMPs), including MMP-2 and MMP-9. The inducible iNOS class of enzyme produces nitric oxide (NO) in high amounts and at a much higher rate. More importantly, elevated levels of nitric oxide can be cytotoxic and will breakdown in the presence of oxygen radicals to form the toxic peroxynitrite (ONOO⁻) radical. There is evidence suggesting that tissue and organ damage, particularly to the brain, lung, and heart, are associated with CABG surgery and are the result of inflammatory processes leading to the production of nitric oxide and its toxic metabolites.

Furthermore, MMPs are known to play a role in tissue re-modeling after a cardiac bypass surgery, and are also implicated in inflammatory diseases, such as rheumatoid arthritis, are a causative contributory factor in joint erosion, and cancer where they play a role in metastasis. Thus, by inhibiting iNOS and MMP activity and/or removing excess concentration of nitric oxide, organ damage, i.e., to the brain, lung, and heart, which are complications associated with the systemic inflammatory response following CABG, could be prevented.

There exists a need for a treatment or prevention of complications described above, and the present invention addresses these needs by providing such methods by administering metal complex nitric oxide scavenger compounds to patients, thereby affording organ protection following CABG and following inflammatory challenges, e.g., ischemia reperfusion injury, surgical trauma, and circulatory shock.

### SUMMARY OF THE INVENTION

The invention provides a method of modulating inflammation associated with trauma, such as caused by surgery, which comprises administering to a patient in need of such modulation an effective amount of a neutral, anionic, or cationic metal complex of Formula I

[Mₐ(X_{b}L)_{c}Y_{d}Zₑ]ⁿ^{_{±}} Formula I,

where:

M is a ruthenium ion;

X is cation or a mixture of cations;

L is a ligand, or mixture of ligands each containing at least two different donor atoms selected from nitrogen, oxygen and sulphur donor atoms;

Y is a ligand, or a mixture of the same or different ligands each containing at least one donor atom, which donor atom is selected from nitrogen, oxygen, sulphur, carbon, and phosphorus, provided that the ligand is not a sulphoxide group, 1,10-phenanthroline or substituted 1,10-phenanthroline, substituted 2,2-bypyridine or dihydrophenazine; and

Z is a halide or pseudohalide ion or a mixture of halide ions and pseudohalide ions;

a=1-3; b=0-12; c=0-18; d=0-18; e=0-18; and n=1-10;

provided that at least one of c, d and e is 1 or more;

and where c is 0; b is also 0;

and where a is 1; c, d and e are not greater than 9 in total;

and where a is 3; c, d and e are not greater than 18 in total;

and where d is 3, Y is not a basic heterocyclic compound containing at least one nitrogen atom.

Also provided is a method of modulating matrix metalloproteinase activity which comprises administering to a patient in need of such modulation an effective amount of a neutral, anionic, or cationic metal complex of the Formula I

[Mₐ(X_{b}L)_{c}Y_{d}Zₑ]ⁿ^{_{±}} Formula I,

where:

M is a ruthenium ion;

X is cation or a mixture of cations;

L is a ligand, or mixture of ligands each containing at least two different donor atoms selected from nitrogen, oxygen and sulphur donor atoms;

Y is a ligand, or a mixture of the same or different ligands each containing at least one donor atom, which donor atom is selected from nitrogen, oxygen, sulphur, carbon, and phosphorus, provided that the ligand is not a sulphoxide group, 1,10-phenanthroline or substituted 1,10-phenanthroline, substituted 2,2-bypyridine or dihydrophenazine; and

Z is a halide or pseudohalide ion or a mixture of halide ions and pseudohalide ions;

a=1-3; b=0-12; c=0-18; d=0-18; e=0-18; and n=1-10;

provided that at least one of c, d and e is 1 or more;

and where c is 0; b is also 0;

and where a is 1; c, d and e are not greater than 9 in total;

and where a is 3; c, d and e are not greater than 18 in total;

and where d is 3, Y is not a basic heterocyclic compound containing at least one nitrogen atom.

Further provided by this invention is a method of improving vascular or haemodynamic function associated with trauma which comprises administering to a patient in need of such improvement an effective amount of a neutral, anionic, or cationic metal complex of Formula I

[Mₐ(X_{b}L)_{c}Y_{d}Zₑ]ⁿ^{_{±}} Formula I,

where:

M is a ruthenium ion;

X is cation or a mixture of cations;

L is a ligand, or mixture of ligands each containing at least two different donor atoms selected from nitrogen, oxygen and sulphur donor atoms;

Y is a ligand, or a mixture of the same or different ligands each containing at least one donor atom, which donor atom is selected from nitrogen, oxygen, sulphur, carbon, and phosphorus, provided that the ligand is not a sulphoxide group, 1,10-phenanthroline or substituted 1,10-phenanthroline, substituted 2,2-bypyridine or dihydrophenazine; and

Z is a halide or pseudohalide ion or a mixture of halide ions and pseudohalide ions;

a=1-3; b=0-12; c=0-18; d=0-18; e=0-18; and n=1-10;

provided that at least one of c, d and e is 1 or more;

and where c is 0; b is also 0;

and where a is 1; c, d and e are not greater than 9 in total;

and where a is 3; c, d and e are not greater than 18 in total;

and where d is 3, Y is not a basic heterocyclic compound containing at least one nitrogen atom.

### BRIEF DESCRIPTION OF THE DRAWINGS

FIG. 1. Illustrates the protocol followed by four groups of dogs. Animals were randomized to receive cardiopulmonary surgery (CPB-PLAC or CPB-SCV) or to serve as controls (CTRL-PLAC or CTRL-SCV). (CPB=cardiac pulmonary bypass; PLAC=placebo; SCV=scavenger; CTRL=control.)

FIGS. 2 and 3a and b. Illustrate mean±SD (standard deviation) values of Ca²⁺-dependent and Ca²⁺-independent NOS activity. Values are shown for left atrium, left ventricle. Lung and brain. * represents a significant difference (p<0.05) between Bypass (black bars) and Control (white bars) groups. Note that Ca²⁺-independent NOS activity is markedly increased in the Bypass group.

FIGS. 4 and 5a and b. Illustrate mean±SD values of matrix metalloprotease MMP activity. Values are shown for left atrium, left ventricle, lung, and brain. *represents a significant difference (p<0.05) between Bypass (black bars) and Control (white bars) groups.

### DISCLOSURE OF THE INVENTION

Nitric oxide plays a varied and vital role in the human body. For example, NO plays a vital role in the control of blood pressure: it acts as a neurotransmitter; and it plays a role in inhibition of platelet aggregation (important in thrombosis or blockages of the blood vessels) and in cytostasis (important in fighting of tumors). Overproduction of NO however, has been implicated in a number of disease states, including vascular/pressor diseases, such as septic shock, post-ischemic cerebral damage, migraine and dialysis induced renal hypotension; immunopathologic diseases, such as hepatic damage in inflammation and sepsis allograft rejection, graft versus host diseases, diabetes and wound healing; neurodegenerative diseases such as cerebral ischemia, trauma, chronic epilepsy, Alzheimer's disease, Huntington's disease, and AIDS dementia complex; and side effects of treatment such as restenosis following angioplastic treatment and secondary hypotension following cytokine therapy. Pharmacological modulation of nitric oxide or other reactive oxygen species in any of these disease states is beneficial.

One above-mentioned disease relating to overproduction of NO is septic shock. This is precipitated by local septicemia or endotoxaemia, (high local levels of bacterial endotoxins). The result is activation of macrophages, lymphocytes, endothelial cells and other cell types capable of producing NO further mediated by cytokine production by these cells. The activated macrophages produce excess NO which causes vasodilatation of the blood vessels, and results in local vascular damage and vascular collapse. This destruction of vascular integrity may be so great that it leads to the collapse of hemodynamic homeostasis, the end result being death.

Current ideas for pharmacological modulation of nitric oxide in such diseases are based on dealing with the mediators of septic shock such as cytokines, endotoxins and platelet activating factor (PAF). The approaches include use of antibodies to cytokines such as tumour necrosis factor (TNF) receptor antagonists such as interleukin I (IL-1) antibodies to lipopolysaccharide (the endotoxins produced by Gram negative bacteria) and PAF antagonists. All such approaches while challenging a factor mediating septic shock do not attempt to deal with the etiology or cause of the disease. Recent advances in understanding of NO have lead to the proposal that inhibitors of the NO synthase enzyme such as N^{G}-monomethy-L-arginine (L-NMMA) may be useful in the treatment of septic shock and other NO overproduction related to diseases since they inhibit NO production. While these inhibitors have shown some utility in animal models and preliminary clinical studies they have the disadvantage of undesirably inhibiting total NO synthesis in the body.

The present invention provides methods for modulating inflammation associated with trauma comprising administering to a patient in need of such modulation an effective amount of a neutral, anionic or cationic metal complex of Formula I. Typically, the inflammation is the result of organ or tissue trauma associated with surgery, such as cardiac bypass surgery, cardiac valve surgery, chronic congestive heart failure, heart transplantation, ischemic reperfusion, angioplasty and the like.

Modulation of the inflammation reduces the tissue damage and/or causes a protective effect against the impairment of an organ function, wherein the organ is the brain, lung, or cardiac muscle. CABG negatively affects cognitive function, including short- and long-term cognitive decline, as well as reduced levels of overall cognitive functioning.

A moderate degree of lung dysfunction is common after CABG surgery, which may contribute to morbidity and mortality. The inflammatory response induced by cardiac surgery leads to activation of complement and influx of granulocytes into the pulmonary vasculature, with increased adherence of neutrophils. Modulation of the inflammatory response by scavenging NO may ameliorate this lung dysfunction.

Following CABG surgery there is a reversible diastolic dysfunction or a reversible depression of systolic function. Nitric oxide has been implicated in cardiac impairment following reperfusion injury, an event occurring post surgery. Increased NO production and interaction with Reactive Oxygen Species (ROS) lead to peroxynitrite production (ONOO⁻). Scavenging of NO could therefore attenuate the production of peroxynitrite. Also NO can depress cardiac muscle contractility thus impairing cardiac function. Increased MMP activity has also been associated with a decrease in cardiac function. The modulation of MMP activity therefore could improve cardiac function after CABG surgery. Scavenging of NO could therefore reduce cardiac dysfunction seen after CABG surgery.

Also provided by the present invention are methods for modulating MMPs utilizing a metal complex of Formula I. MMPs are a family of zinc-containing enzymes that are involved in the remodeling and degradation of extracellular matrix proteins. (See A. Zask, et al., Current Pharmaceutical Design, (1996), 2, 624-661.) The natural substrates for MMPs are a family of proteins, including collagen, gelatin, laminin, fibronectin and elastin, located in the extracellular matrix (ECM). (See D. E. Levy, et al., “Chapter Ten: Matrix Metalloproteinase Inhibitor Drugs”, Emerging Drugs, (1997), 2, 205-230; and D. Celentano, et al., J. Clin. Pharmacol., (1997), 37, 991-1000.)

The aberrant regulation of MMP production has been implicated in pathological tissue breakdown in numerous disease processes. Increased levels of MMPs are associated with cardiovascular diseases, including, but not limited to, smooth muscle cell migration in restenosis, and plaque formation and rupture in atherosclerosis. MMPs are implicated in angiogenesis, vascular diseases, autoimmune diseases, such as multiple sclerosis (MS), and the like, rheumatoid arthritis (RA), inflammatory bowel disease (IBD), osteoporosis, renal disease, tissue ulceration, retinopathy, corneal ulceration, psoriasis, periodontal disease, abnormal wound healing, rupture of fetal membranes, tumor growth, and tumor metastasis.

Additional inflammatory diseases associated with MMPs include, but are not limited to, systemic lupus erythematosis, acute transplantation/graft rejection, myasthenia gravis, progressive systemic sclerosis, multiple myeloma, atopic dermatitis, hyperimmunoglobin E, hepatitis B antigen, negative chronic active hepatitis, Hashimoto's thyroiditis, Familial Mediterranean fever, Grave's disease, autoimmune hemolytic anemia, and primary biliary cirrhosis.

MMPs have similar domain structures with four major regions: an N-terminal leader sequence involved in secretion, a prodomain that inhibits the enzymatic activity, a catalytic domain, a hemopexin domain involved in substrate specificity and enzyme activity. MMP subtypes include: MMP-1, MMP-8, MMP-13, MMP-7, MMP-3, MMP-10, MMP-11, MMP-12, MMP-2, MMP-9, MMP-14, MMP-15, MMP-16 and MMP-17. Typically the MMP family is subdivided based upon substrate preferences as: collagenases, gelatinases, stromelysins, and integral membrane proteins.

The metal complexes of the present invention can be utilized in conjunction with other therapies, such that the dosage required for the additional therapy is reduced. For example, in the instance of a vascular disease treatment or therapy, the dosage could be reduced. Alternatively, the amount of fluid introduced also can be reduced. The applicable vasoconstrictor or fluid is administered in an ICU setting to restore or maintain blood pressure.

The present invention also provides methods of improving vascular or hemodynamic function associated with surgical trauma by administering a metal complex of Formula I. These functions relate to circulation of the blood and include, but are not limited to, blood vessel integrity, reversal of NO mediated hypotension, maintenance of vascular tone, maintenance of blood pressure, reversal of hyporesponsiveness to vasoconstrictors, reduction of vascular leak (blood vessel integrity), and improvement in the regulation of microcirculation through organs including (but not restricted to) heart, lung, kidney, and liver.

Some metal complexes are known in pharmaceutical compositions for the treatment of diseases of the human body. For example certain complexes of platinum and ruthenium have been used or indicated in the treatment of cancer. This invention provides for the use of a neutral anionic or cationic metal complex having at least one site for coordination with NO of Formula I

[Mₐ(X_{b}L)_{c}Y_{d}Zₑ]ⁿ^{_{±}} Formula I

in the manufacture of a medicament for the attenuation of NO levels and other reactive oxygen species when implicated in disease,

where:

M is a metal ion or a mixture of metal ions;

X is a cation or a mixture of cations;

L is a ligand, or mixture of ligands each containing at least two different donor atoms selected from the elements of Group IV, Group V or Group VI of the Periodic Table;

Y is a ligand or a mixture of the same or different ligands each containing at least one donor atom or more than one donor atom which donor atom is selected from the elements of Group IV, Group V or Group VI of the Periodic Table; and

Z is a halide or pseudohalide ion or a mixture of halide ions and pseudohalide ions;

a=1-3; b=0-12; c=0-18; d=0-18; e=0-18; and n=0-10; provided that at least one of c, d and e is 1 or more;

and where c is 0: b is also 0;

and where a is 1: c, d and e are not greater than 9;

and where a is 2: c, d and e are not greater than 12. By “complex” in this specification is meant a neutral complex or anionic or cationic species.

The term “Group” which is used herein is to be understood as a vertical column of the periodic table in which elements of each Group have similar physical and chemical properties. The definition of the Periodic Table is that credited to Mendeleev; Chamber Dictionary of Science and Technology, 1974 Published by W & R Chambers Ltd.

This invention may also be stated as providing a method of attenuation of reactive oxygen species when implicated in diseases of the human body. Thus the invention comprises administering a pharmaceutical composition containing a neutral, anionic or cationic metal complex of Formula I.

This invention may also provide for the use of a neutral anionic or cationic metal complex of formula I in the manufacture of a medicament for the treatment of diseases in which reactive oxygen species are overproduced.

This invention may also be stated as providing a method of attenuation of nitric oxide when implicated in diseases of the human body. Thus the invention comprises administering a pharmaceutical composition containing a neutral, anionic or cationic metal complex of Formula I. This invention may also be stated as providing a method of treatment of diseases of the human body resultant of overproduction of NO in the human body comprising administering a pharmaceutical composition containing a neutral anionic or cationic metal complex of Formula I. Where the Formula I represents an anionic species a cation will also be present. Where Formula I represent a cationic species an anion will also be present. The metal complexes may be hydrated. Preferably M is a first, second or third row transition metal ion. For example M may be an Rh, Ru, Os, Mn, Co, Cr or Re ion, and is preferably an Rh, Ru or Os ion.

Suitably M is in an oxidation state III. When the metal ion for example ruthenium is in oxidation state III, the rate at which it binds with NO is significantly faster than when it is in oxidation state II.

X may be any cation, such as mono-, di- or tri-valent cation. Suitable cations may be H⁺, K⁺, Na⁺, NH₄⁺ or Ca²⁺. Conveniently X may be H⁺, K⁺, or Na ⁺.

Preferably L is a ligand, such as ethylenediamine-N,N′-diacetic acid (edda), ethylenediaminetetraacetic acid (edta), nitrilotriacetic acid (nta), dipicolinic acid (dipic), picolinic acid (pic), diethylenetri-aminepentaacetic acid (dtpa), thiobis(ethylenenitrilo)tetraacetic acid (tedta), dithioethanebis(ethylenenitrilo)tetraacetic acid (dtedta), and N-(2-hydroxyethyl) ethylenediamine-triacetic acid (hedtra).

Preferably Y is a ligand containing nitrogen, oxygen, sulphur, carbon or phosphorus donor groups. Suitable nitrogen donor groups may be for example ammine, amine, nitrile and nitride or derivations thereof. Suitable oxygen donor groups may be for example carboxylic acid, ester or derivations thereof, water, oxide, sulphoxide, hydroxide, acetate, lactate, propionate, oxalate and maltolate. Suitable sulphur donor groups may be for example sulphoxide, dialkysulphide, dithiocarbamate or dithiophosphate. Suitable carbon donor groups may be for example carbon monoxide or isocyanide. Suitable phosphorus donor groups may be for example trialkylphosphine.

Z may be any halide and is preferably chloride, bromide or iodide. Most conveniently, Z is chloride.

Examples of metal complexes for use according to the present invention include optionally hydrated ruthenium complexes of Formula II

[Ru(H₀₋₆L^{II})₁₋₃Y₀₋₂Cl₀₋₄]^{(0-4)±} Formula II

where L^{II} is a polydentate aminocarboxylate ligand, for example edta, nta, dipic, pic, edda, tropolone, dtpa, hedtra, tedta or dtedta or diamide of edta or dtpa (or an amide or ester derivative thereof) or a mixture of any of these and Y is as defined above and may for example be selected from: acetylacetone (acac); a β-diketonate; water; dimethylsulphoxide (dmso); carboxylate; bidentate carboxylate; catechol; kojic acid; maltol; hydroxide; tropolone; malonic acid; oxalic acid; 2.3-dihydroxynaphthalene; squaric acid; acetate; a sulphate and a glycolate. The skilled artisan will be able to substitute other known ligands at Y and which will fall within the scope of the inventions.

Preparative methods of tedta, dtedta and diamide of edta and dtpa are described in the following references respectively:

P Tse & J E Powell, Inorg Chem, (1985), 24, 2727;

G Schwartzenbach, H Senner, G Anderegg, Helv Chim Acta (1957), 40, 1886;

M S Konings, W C Dow, D B Love, K N Raymond, S C Quay and S M Rocklage, Inorg Chem (1990), 29, 1488-1491; and

P N Turowski, S J Rodgers, R C Scarrow and K N Raymond, Inorg Chem (1988), 27, 474-481.

Where the complex of Formula II is an anion, a cation will be required. For example the complexes of Formula II are present in

K[Ru(Hedta)Cl]2H₂O

[Ru(H₂edta)(acac)]

K[Ru(hedtra)Cl]H₂O

K[Ru(dipic)₂]H₂O

(H₂pic)[RuCl₂(pic)₂](Hpic)H₂O

K[Ru(H₂edta)Cl₂]H₂O

K[Ru(Hnta)₂]½H₂O

K[Ru(H₂dtpa)Cl]H₂O

[Ru(Hhedtra)acac]H₂O

[Ru(Hhedtra)trop] or

[Ru(H₃dtpa)Cl].

Complexes of Formula H have not to the best of our knowledge been previously indicated in any pharmaceutical composition. Therefore the present invention also provides a pharmaceutical composition containing an optionally hydrated ruthenium complex of Formula II.

Further examples of metal complexes for use according to the present invention include optionally hydrated complexes of Formula III,

[M₁₋₃Y₁₋₁₈Cl₁₀₋₁₈]^{(0-6)±} Formula III

where Y is a sulphur donor ligand. For example, such complex is present in

[Ru(mtc)₃] (mtc=4-morpolinecarbodithoic acid) or Ru(S₂CNCH₂CH₂NMeCH₂CH₂)₃½H₂O.

Complexes of Formula III in which Y is a sulphur donor ligand have not to the best of our knowledge been previously indicated in any pharmaceutical composition. Therefore, the present invention also provides a pharmaceutical composition containing an optionally hydrated complex of Formula III when Y is a sulphur donor ligand.

Yet further examples of metal complexes for use according to the present invention include optionally hydrated complexes of ruthenium of Formula IIIa

[M^{III}₁₋₃Y^{III}₁₋₁₈Cl₀₋₁₈]^{(0-6)±} Formula III

where M^{III} is ruthenium and Y^{III} is an oxygen-donor ligand such as acetate, lactate, water, oxide, propionate (COEt), oxalate (ox), or maltolate (maltol) or a combination of these. For example complexes of Formula III or IIIa are present in

[Ru₃O(OAc)₆](OAc)

[Ru₃O(lac)₆](lac)

[Ru₂(OAc)₄]NO₃

[Ru₂(OCOEt)₄]NO3

K₃[Ru(ox)₃]

[Ru₂(OAc)₄]Cl or

[Ru(maltol)₃].

Some complexes of Formula III have not to the best of our knowledge been previously indicated in any pharmaceutical composition. Therefore the present invention also provides a pharmaceutical composition containing an optionally hydrated complex of ruthenium of Formula III wherein M^{III} is ruthenium and Y^{III} is an oxygen-donor ligand selected from the group acetate, lactate, oxide, propionate and maltolate.

Further examples of metal complexes for use according to the present invention include optionally hydrated complexes of ruthenium of Formula IV

[RuY^{IV}₁₋₉Cl₁₋₉]^{(0-4)±} Formula IV

where Y^{IV} is a nitrogen-donor ligand such as: ammine; ethylenediamine (en); pyridine (py); 1,10-phenanthroline (phen): 2,2-bipyridine (bipy) or 1,4,8,11-tetraazacyclotetradecane (cyclam); 2,3,7,8,12,13,17,18-octaethylporphyrin (oep); or a combination of these. For example complexes of Formula IV are present in

[Ru(HN₃)₅Cl]Cl₂

trans-[RuCl₂(py)₄]

K[Ru(phen)Cl₄]

[Ru(cyclam)Cl₂]Cl

K[Ru(bipy)Cl₄]

[Ru(NH₃)₆]Cl₃ or

[Ru(NH₃)₄Cl₂]Cl.

Some complexes of Formula IV have not to the best of our knowledge been previously indicated in any pharmaceutical composition. Therefore the present invention also provides a pharmaceutical composition containing an optionally hydrated complex of ruthenium of Formula IV wherein Y^{IV} is a nitrogen-donor ligand selected from the group en, py, phen, bipy, cyclam and oep. Derivations of these ligands can be prepared by a skilled artisan and which will fall within the scope of the inventions.

Still further examples of metal complexes for use according to the present invention include optionally hydrated complexes of ruthenium or osmium of general Formula V

[M₁₋₃Y^{V}₁₋₁₈Cl₀₋₁₈]^{(0-6)±} Formula V

where Y^{V} is a combination of donor ligands such as are described hereinabove, for example ammine, dmso, oxalate, bipy, acac and acetonitrile (MeCN). Complexes of Formula V are present in for example

[Ru(NH₃)(dmso)₂Cl₃]

cis-[Ru(dmso)₄Cl₂]

cis-[Ru(NH₃)(dmso)₃Cl₂]

[Ru(dmso)₃Cl₃]

[Os(ox)(bipy)₂]H₂O or

[Ru(acac)₂(MeCN)₂]CF₃SO₃.

The complex ions of the latter two compounds above have not to the best of our knowledge been previously indicated in any pharmaceutical composition. Therefore the present invention also provides a pharmaceutical composition containing an optionally hydrated complex of formula [Os(ox)(bipy)₂]; and further a pharmaceutical composition containing an optionally hydrated complex of formula [Ru(acac)₂(MeCN)₂]⁺.

The complexes of the present invention may be included as an active component in a pharmaceutical composition containing an optionally hydrated complex of any of Formulae I-V, in admixture with a pharmaceutically acceptable carrier or diluent. Said pharmaceutical composition may be formulated according to well known principles, and may be in the form of a solution or suspension for parenteral administration in single or repeat doses or be in capsule, tablet, dragee, or other solid composition or as a solution or suspension for oral administration, or formulated into pessaries or suppositories, or sustained release forms of any of the above. The solution or suspension may be administered by a single or repeat bolus injection or continuous infusion, or any other desired schedule. Suitable diluents, carriers, excipients and other components are known. Said pharmaceutical composition may contain dosages determined in accordance with conventional pharmacological methods, suitable to provide active complexes in the dosage range in humans of 1 mg to 10 g per day. Actual required dosage is largely dependent on where in the body there is the excess concentration of NO or other reactive oxygen species and for how long overproduction continues or attenuation of the levels of NO or reactive oxygen species, where such reactive oxygen species is implicated in disease, is required.

The model of CABG used in this invention differs in some aspects from the human heart model. For this invention, the use of the dog to represent the human model is described, where the results are representative of human CABG procedure. Dogs, unlike human, do not have pre-existing heart disease. Intraoperative bypass grafting was not performed and aortic flow was occluded using an internal rather than an external cross-clamp. However, it has been previously shown that sham bypass can activate inflammatory pathways (Mayers et al. J. Crit. Care. 11:189-197 (1996)). Therefore, the control animals were anesthetized but not subject to a sham bypass procedure or re-infusion of blood collecting within the mediastinum.

### EXAMPLES

Having now generally described the invention, the same will be more readily understood through reference to the following examples which are provided by way of illustration, and are not intended to be limiting of the present invention, unless specified.

### Example A

Animal Preparation

These studies were carried out with the approval of the University of Saskatchewan Animal Care Committee and comply with published National Institutes of Health guidelines. Mongrel dogs (weight 20-25 kg) were anesthetized with pentobarbital (15 mg/kg), incubated and mechanically ventilated (Harvard Model #607). Anesthesia was maintained by continuous infusion of pentobarbital (1.0 mgrkg/hr), morphine (0.1 mg/kg/hr), and vecuronium (0.1 mg/kg/hr). A pulmonary artery catheter was used to measure mean pulmonary artery pressure (P_{PA}), pulmonary capillary wedge pressure (P_{CWP}), mean right atrial pressure (P_{RA}), and cardiac output (CO). Mean systemic artery pressure (P_{SA}) and arterial blood gas sampling were obtained through a femoral artery catheter.

The CPB animal preparation has been previously described (Mayers et al., J. Thoracic Cardiovasc. Surg. 117:1009-1016, 1999). Briefly, the heart was exposed via midline sternotomy. Following heparinization (5,000 U i.v. followed by 1,000 units/hr i.v.), catheters were placed in the left atrium and right atria to complete the bypass circuit. A balloon angioplasty catheter (#6 F), positioned proximal to the aortic valve via the right internal carotid artery, acted to internally cross-clamp the aorta when inflated. CPB was initiated using a membrane oxygenator (Capiox Hollow Fiber Oxygenator) and a blood pump (Sarns Model 5000 Console) at 100 ml/kg flow. Cold (7-8° C.), antegrade cardioplegia solution was delivered and then the aorta was occluded by inflation of the angioplasty balloon. Blood cardioplegia (BCD4 Sharely) was continuously administered to eliminate electrical activity. The animals were cooled (24° C.) and the aortic occlusion maintained for a further 50 minutes. Then the angioplasty balloon was deflated, mechanical ventilation resumed and the animals warmed over 30 minutes. Therefore, CPB lasted for a total of 90 minutes. P_{SA} was prospectively maintained at >60 mmHg by first increasing P_{RA} to 15 mmHg by fluid administration and then infusing phenylephrine. The animals were maintained for 4 hours post-CPB.

Experimental Groups

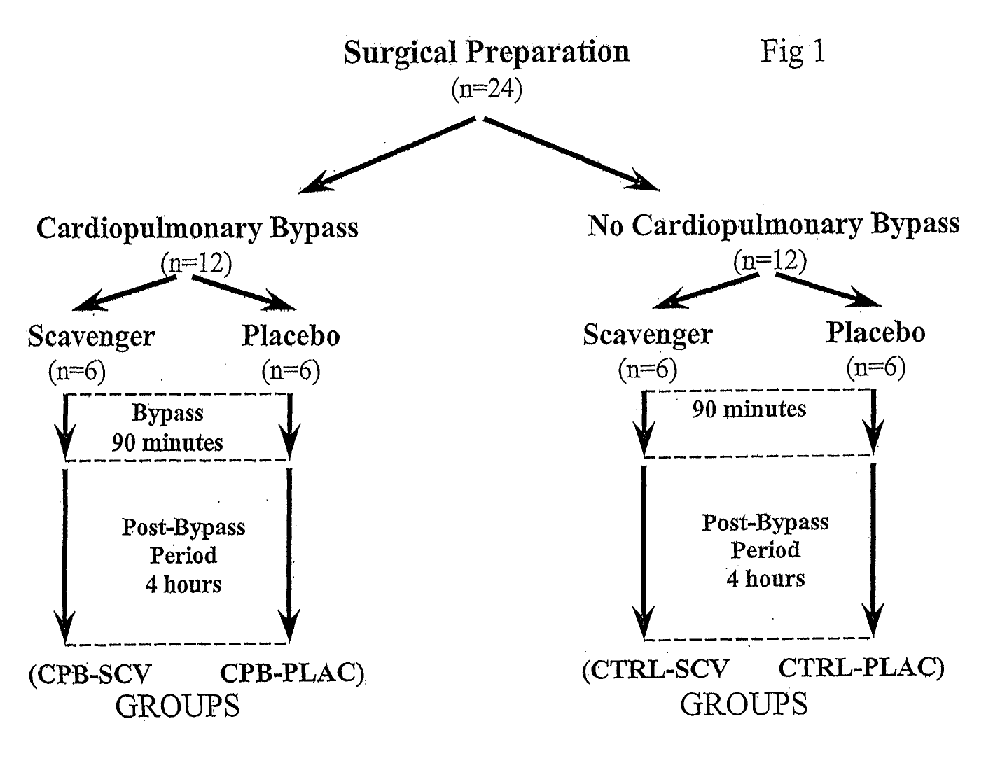

Dogs were randomized to receive CPB (n=12) or act as controls (n=12). Dogs receiving CPB were then randomized to receive a continuous infusion of an NO scavenger [Ru(H₃dtpa)]Cl (CPB-SCV group; n=6) or to receive a placebo (CPB-PLAC group; n=6). The NO scavenger (6221 AnorMED) was continuously infused (128 mg/kg/hr) starting prior to the sternotomy and ending 30 minutes following termination of CPB (see FIG. 1). This dose was chosen based upon pilot data from endotoxin treated (0.2 mg/kg) dogs to simulate a state of NO excess (data not shown). Arterial pH was maintained between 7.3 and 7.45 by NaHCO₃ as needed.

The control dogs followed a similar protocol but did not have a sternotomy or placement of CPB catheters. At the conclusion of the studies all animals were sacrificed by barbiturate overdose. Biopsies (0.5 cm²) from left ventricle, left atrium, lung (right lower lobe) and brain (cerebral cortex) were obtained, frozen in liquid nitrogen, and later analyzed (see below).

Measurements and Calculations

After catheter insertions, baseline blood samples were obtained for routine hematology, biochemistry and blood gases (see Table 1). Hemodynamics measurements including SVR, fluid requirements, CO, P_{SA}, P_{CWP}, and P_{RA} were repeated at 1 and 4 hours following bypass and as needed to maintain our predetermined hemodynamic goals post CPB. Neutrophil expression of CD18 was assessed with flow cytometry (FACScan) as previously described (Mayers et al., J. Crit. Care. 1996; 11:189-197). Briefly, after cells were prepared they were reacted with an irrelevant, isotype-matched FITC-conjugated rat monoclonal antibody (MCA1125F)(Serotech Ltd) or FITC-conjugated rat anti-human CD18 antibody (MCA503F; Serotech) that cross-reacted with canine. Flow cytometric analysis was restricted to neutrophils based upon forward angle and right angle light scatter. Neutrophil CD18 expression was determined as the percentage of cells with a fluorescence exceeding that of cells reacted with the irrelevant, isotype-matched control.

NOS activity was measured in homogenized tissue samples as previously described (Radomski et al., Cardiovasc Res. 1993; 27:1380-1382). Briefly, tissue aliquots were incubated with L[U¹⁴-C]-arginine (Amersham) for 20 min in the appropriate buffer. NOS-dependent L-citrulline formation, expressed as pmol/min/mg protein, was used as an index of enzyme activity. A Ca²⁺-chelating agent (EGTA, 1 mM) was used to differentiate between Ca²⁺-dependent and -independent NOS activities of enzymes. Tissue MMPs activity was measured by zymography as previously described (Sawicki et al., Nature 1997; 386:616-618) using 7% SDS-PAGE with copolymerized gelatin as a substrate.

Statistics

Data were compared between period and groups with a one way or two-way analysis of variance as appropriate and when the F statistic showed a significant difference, a SNK (Student-Newman-Keuls test) multiple comparison test was used to determine specific group and period differences. We prospectively limited the possible number of comparisons to limit required correction factor. Spearman correlation was used to assess the interaction between CD1 8 expression and NOS or MMP activity. A p value less than 0.05 was considered to show a significant difference. All values are shown as means±SD.

### RESULTS

Hemodynamics and Gas Exchange

P_{SA} and CO decreased while systemic vascular resistance (SVR) increased over time in the CPB groups (Table 1). To achieve our predefined hemodynamic endpoints (P_{SA}>60 mm Hg and P_{RA}<15 mm Hg), 3 of 6 CPB-PLAC animals required a phenylephrine compared to 0 of 6 the CPB-SCV animals (p<0.05). Total fluid administration was reduced in the NO scavenger groups compared to the placebo groups.

Pooled CK values (Table 2) in the CPB-PLAC group were increased over the CPB-SCV group (2654±6225 U vs 476±681 U; p<0.005). Comparing baseline values to 4 hr post CPB, PO₂ decreased in the CPB-SCV group and CPB-PLAC group and intrapulmonary shunt increased (19%±10 versus 18%±14 respectively; p>0.05). Neutrophils, as apercentage of WBC, increased from Baseline to 4 hr post CPB in the CPB-PLAC group (66%±7 to 83%±5; p=0.03). CD18 expression at 4 hr post CPB was lower in CPB-SCV group compared to the CPB-PLAC group (p<0.05).

NOS and MMP Activities

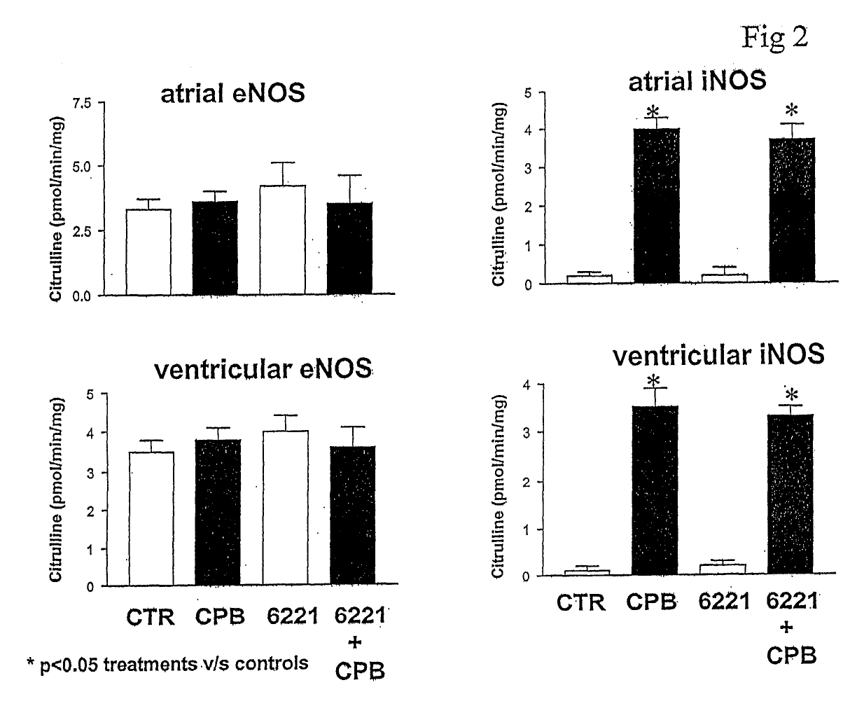

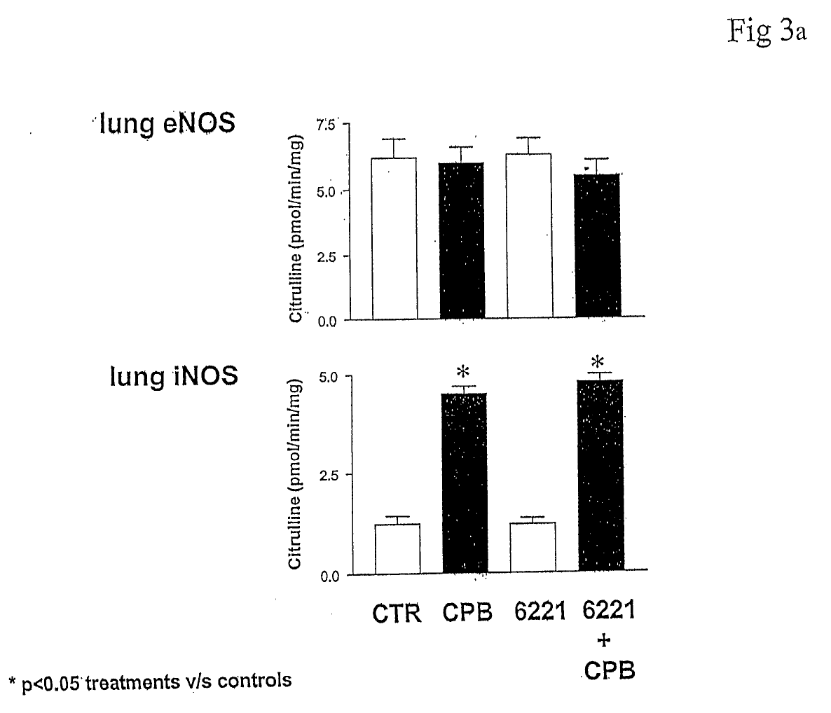

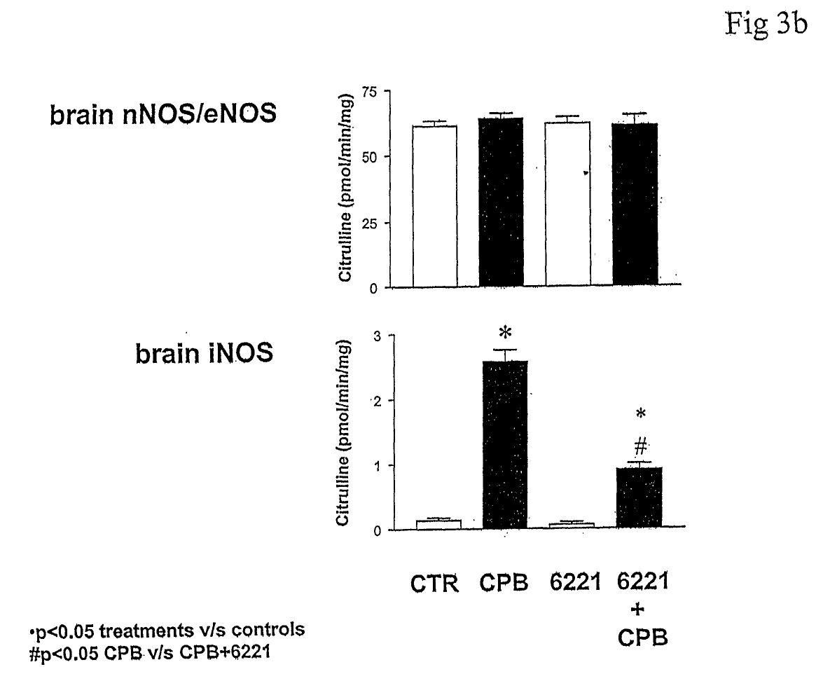

Ca²⁺-dependent NOS activities were similar between all groups in heart, brain and lung tissues. Ca²⁺-independent NOS activities were elevated (p<0.05) in the both CPB groups compared to both CTRL groups (FIG. 2 and FIG. 3). Brain Ca²⁺-independent NOS activity was significantly reduced (p<0.05) in the CPB-SCV group compared with CPB-PLAC group but was still increased compared with the groups that did not receive CPB (p<0.05).

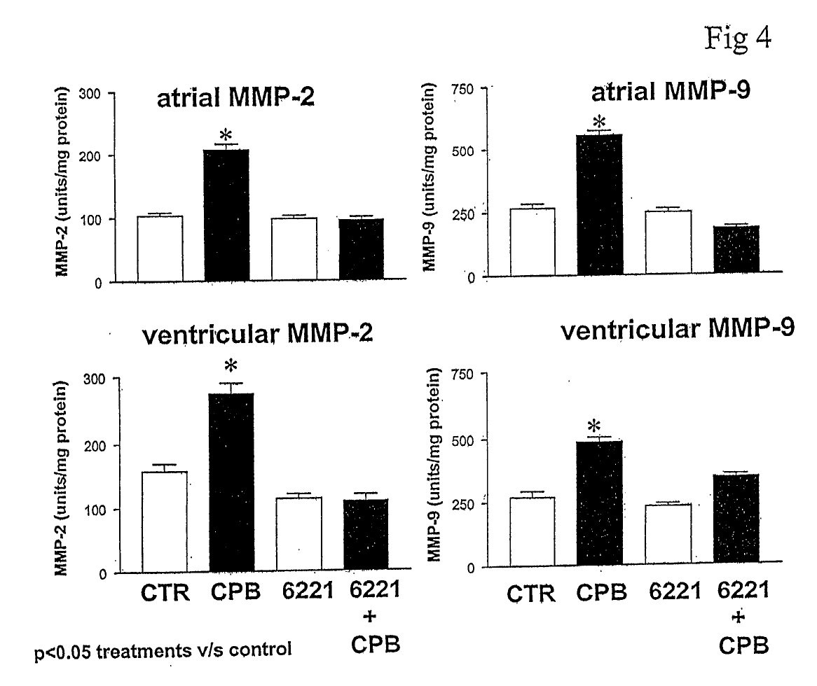

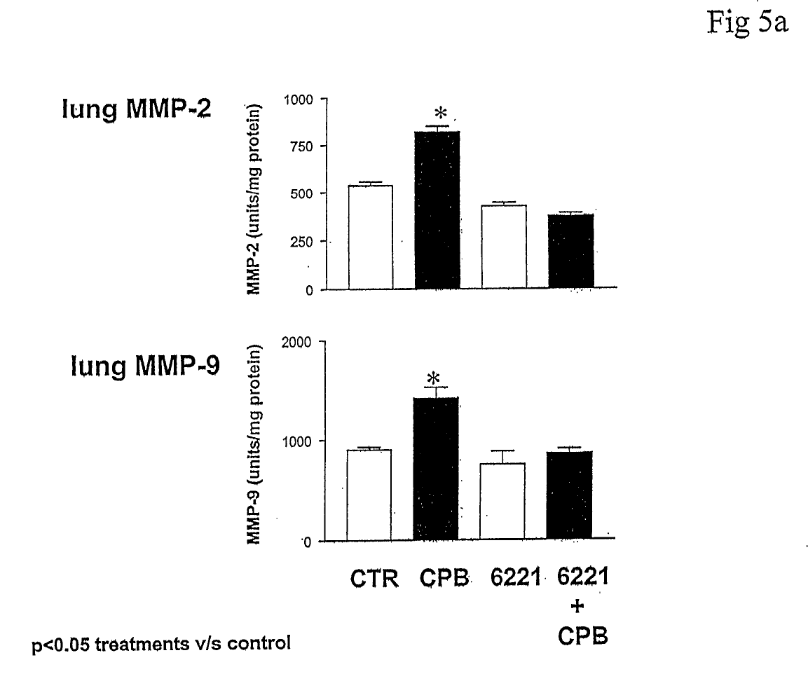

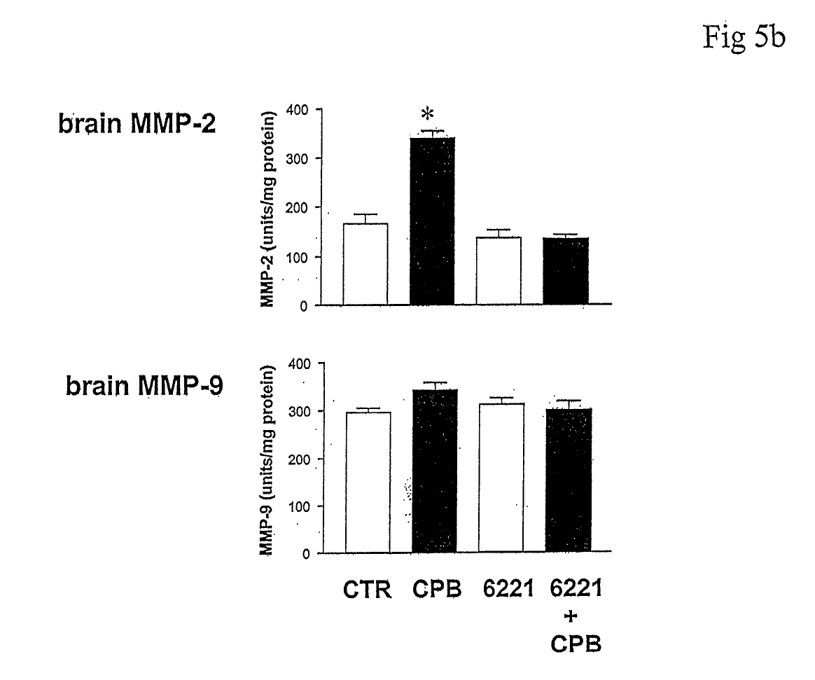

MMP-2 and MMP-9 activities were significantly (p<0.05) elevated in the CPB-PLAC group compared to CRTL-PLAC group in heart and lung (FIG. 4). Only MMP-2 was significantly (p<0.05) elevated in brain in the CPB-PLAC group compared to the CRTL-PLAC group (FIG. 5). MMP activity was significantly reduced in the CPB-SCAV group compared to the CPB-PLAC group (p<0.05). MMP activity, except brain MMP-9, was positively correlated to CD18 peak expression (rho range from 0.46 to 0.73; p<0.005). NOS activity was not correlated to CD18 peak expression (p>0.05).

TABLE 1
Selected hemodynamic values.

| | Baseline | 1 Hr Post | 4 Hr Post |
|---|---|---|---|
| P_{SA} | | | |
| (mm Hg) | | | |
| CTRL-PLAC | 123 ± 15 | 112 ± 15 | 109 ± 12 |
| CTRL-SCV | 106 ± 16 | 114 ± 14 | 113 ± 17 |
| CPB-PLAC | 125 ± 11 | 98 ± 14* | 101 ± 18* |
| CPB-SCV | 142 ± 9 | 89 ± 13* | 95 ± 4* |
| P_{RA} | | | |
| (mm Hg) | | | |
| CTRL-PLAC | 6 ± 2 | 7 ± 2 | 8 ± 2 |
| CTRL-SCV | 5 ± 1 | 7 ± 2 | 6 ± 2 |
| CPB-PLAC | 5 ± 1 | 6 ± 1 | 6 ± 1 |
| CPB-SCV | 6 ± 1 | 8 ± 2 | 7 ± 2 |
| P_{CWP} | | | |
| (mm Hg) | | | |
| CTRL-PLAC | 8 ± 2 | 8 ± 1 | 9 ± 1 |
| CTRL-SCV | 6 ± 1 | 7 ± 1 | 6 ± 1 |
| CPB-PLAC | 8 ± 2 | 9 ± 2 | 8 ± 2 |
| CPB-SCV | 9 ± 2 | 9 ± 1 | 9 ± 3 |
| SVR | | | |
| (dyne/sec/cm⁵) | | | |
| CTRL-PLAC | 6503 ± 1517 | 5461 ± 1191 | 6347 ± 1025 |
| CTRL-SCV | 8403 ± 1360 | 9452 ± 2264 | 11143 ± 2130*# |
| CPB-PLAC | 8112 ± 2251 | 6898 ± 1323 | 11672 ± 918* |
| CPB-SCV | 9593 ± 2713 | 7227 ± 2062 | 11406 ± 2812* |
| CO | | | |
| (L/min) | | | |
| CTRL-PLAC | 1.5 ± 0.4 | 1.6 ± 0.6 | 1.4 ± 0.2 |
| CTRL-SCV | 1.0 ± 0.3 | 1.0 ± 0.2 | 0.8 ± 0.2 |
| CPB-PLAC | 1.3 ± 0.4 | 1.1 ± 0.3 | 0.7 ± 0.2* |
| CPB-SCV | 1.2 ± 0.5 | 1.0 ± 0.2 | 0.7 ± 0.1* |
| Fluid | | | |
| Requirements | | | |
| (ml) | | | |
| CTRL-PLAC | — | — | 2655 ± 82+ |
| CTRL-SCAV | — | — | 892 ± 159 |
| CPB-PLAC | — | — | 1617 ± 254+ |
| CPB-SCV | — | — | 983 ± 134 |

Where
P_{SA} = mean systemic blood pressure,
P_{RA} = mean right atrial pressure,
P_{CWP} = pulmonary capillary wedge pressure,
SVR = systemic vascular resistance (calculated as SVR = Psa-Pra/CO *80),
CO = cardiac output.
Note
*denotes a significant difference (p < 0.05) compared to other periods within same group,
#denotes a significant difference (p < 0.05) compared to the same period in the placebo group (CTRL-PLAC or CPB-PLAC),
+denotes a significant difference (p < 0.05) comparing pooled groups (CTRL-PLAC vs CTRL-SCV or CPB-PLAC vs CPB-SCV)

TABLE 2
Selected biochemical and hematological values in all four groups.

| | Baseline | 1 Hr Post | 4 Hr Post |
|---|---|---|---|
| Hemoglobin | | | |
| (gm/dL) | | | |
| CTRL-PLAC | 10.9 ± 1.3 | 9.9 ± 1.5 | 9.0 ± 1.2 |
| CTRL-SCV | 11.7 ± 0.7 | 11.4 ± 0.8 | 11.9 ± 0.8*# |
| CPB-PLAC | 12.5 ± 0.8 | 6.7 ± 0.4* | 7.9 ± 0.6* |
| CPB-SCV | 10.4 ± 3.4 | 7.3 ± 0.7* | 7.6 ± 1.1* |
| WBC | | | |
| (10⁹ cells) | | | |
| CTRL-PLAC | 8.1 ± 2.0 | 7.2 ± 1.2 | 7.7 ± 0.8 |
| CTRL-SCV | 6.6 ± 2.3 | 7.7 ± 2.6 | 8.6 ± 2.2 |
| CPB-PLAC | 5.3 ± 1.5 | 5.3 ± 1.7 | 6.9 ± 2.7 |
| CPB-SCV | 6.0 ± 1.4 | 5.4 ± 1.7 | 6.7 ± 1.7 |
| PO₂ | | | |
| (mm Hg) | | | |
| CTRL-PLAC | 569 ± 20 | 557 ± 58 | 589 ± 23 |
| CTRL-SCV | 558 ± 28 | 564 ± 42 | 566 ± 24 |
| CPB-PLAC | 438 ± 63 | 273 ± 168* | 276 ± 135* |
| CPB-SCV | 442 ± 52 | 303 ± 144* | 305 ± 121* |
| PCO₂ | | | |
| (mm Hg) | | | |
| CTRL-PLAC | 39 ± 5 | 41 ± 3 | 40 ± 2 |
| CTRL-SCV | 32 ± 10 | 34 ± 7 | 34 ± 8 |
| CPB-PLAC | 36 ± 7 | 36 ± 4 | 39 ± 5 |
| CPB-SCV | 25 ± 8 | 34 ± 8 | 30 ± 6 |
| CD18 | | | |
| expression | | | |
| (units) | | | |
| CTRL-PLAC | 65 ± 11 | 71 ± 8 | 66 ± 7 |
| CTRL-SCV | 53 ± 25 | 51 ± 31 | 35 ± 26*# |
| CPB-PLAC | 69 ± 22 | 78 ± 15 | 81 ± 11 |
| CPB-SCV | 52 ± 25 | 54 ± 33 | 38 ± 27# |
| CK | | | |
| CTRL-PLAC | 87 ± 42 | 71 ± 69 | 74 ± 58 |
| CTRL-SCV | 97 ± 20 | 132 ± 105 | 88 ± 32 |
| CPB-PLAC | 118 ± 30 | 2858 ± 4976 | 4986 ± 8475 |
| CPB-SCV | 44 ± 34 | 434 ± 513 | 948 ± 800 |

Where
WBC = white blood cell corrected for hemodilution effect,
CD18 expression = peak fluorescent intensity of neutrophils for CD18 (see text).
Note
*denotes a significant difference (p < 0.05) compared to other periods within same group,
#denotes a significant difference (p < 0.05) compared to the same period in the placebo group (CTRL-PLAC or CPB-PLAC),
+denotes a significant difference (p < 0.05) comparing pooled groups (CTRL-PLAC vs CTRL-SCV or CPB-PLAC vs CPB-SCV).
PO₂ = partial pressure of oxygen (this is a measure of the dissolved oxygen concentration),
PCO₂ = partial pressure of carbon dioxide (this is a measure of the dissolved carbon dioxide concentration), and
CK = creatine kinase.

A Ruthenium NO scavenger complex (AMD6221) [Ru(H₃dtpa)Cl)] was administered to dogs undergoing cardiopulmonary bypass. Control, untreated animals required both fluid resuscitation and vasoconstrictors to maintain blood pressure. In addition there was upregulation of iNOS and MMPs in lung, cardiac and brain tissue, and increased levels of circulating neutrophils. In AMD6221 dogs there was a reduced requirement for both fluid resuscitation and vasoconstrictors, which is compatible with previously published data on AMD6245 [Ru(Hedta)H₂O] in septic shock. There was also a reduction in creatine kinase levels in treated animals. These indicators point to an improvement in cardiac function with treatment. MMPs are implicated in cardiac dysfunction, so lowering of MMPs in all tissues provides an organ protective effect of the NO scavenger in the heart, lung and brain. NO is also a mediator of tissue damage and therefore lowering NO levels also will contribute to the protective effect. The reduction in creatine kinase CK levels also indicates a protective effect towards brain and muscle.

A number of commercially available compounds, and compounds prepared by routes known in the literature, containing the complexes of the present invention were tested in vitro, in vitro cell culture, and ex-vivo in order to determine ability to coordinate with NO. The complexes tested were as follows:

TABLE 3

| | | Literature Reference |
|---|---|---|
| Example | Compound | for Preparation |
| 1 | K[Ru(Hedta)Cl]2H₂O | A A Diamantis & J V |
| | | Dubrawski, Inorg. Chem, |
| | | (1981), 20, 1142-50 |
| 2 | [Ru(H₂edta)(acac)] | A A Diamantis & J V |
| | | Dubrawski, Inorg. Chem., |
| | | (1983), 22, 1934-36 |
| 3 | K[Ru(hedtra)Cl]H₂O | H C Bajaj & R van Eldik, |
| | | Inorg. Chem. (1982), 28, |
| | | 1980-3 |
| 4 | K[Ru(dipic)₂]H₂O | N H Williams & J K |
| | | Yandell, Aust. J. Chem. |
| | | (1983), 36 (12), 2377-2386 |
| 5 | (H₂pic)[RuCl₂(pic)₂](Hpic)H₂O | J D Gilbert, D Rose & |
| | | G Wilkinson, J. Chem. Soc. |
| | | (A), (1970), 2765-9 |
| 6 | K[Ru(H₂edta)Cl₂]H₂O | A A Diamantis & J V |
| | | Dubrawski, Inorg. Chem. |
| | | (1981), 20, 1142-50 |
| 7 | K[Ru(Hnta)₂]½H₂O | M M Taqui Khan, |
| | | A Kumar & Z Shirin, |
| | | J. Chem. Research (M), |
| | | (1986), 1001-1009 |
| 8 | K[Ru(H₂dtpa)Cl]H₂O | M M Taqui Khan, |
| | | A Kumar & Z Shirin, |
| | | J. Chem. Research (M). |
| | | (1986), 1001-1009 |
| 9 | [Ru₃O(lac)₆](lac) | A Spencer & G Wilkinson, |
| | | J. Chem. Soc. Dalton |
| | | Trans, (1972), 1570-77 |
| 10 | [Ru₃O(OAc)₆](OAc) | A Spencer & G Wilkinson, |
| | | J. Chem. Soc. Dalton |
| | | Trans. (1972), 1570-77 |
| 11 | [Ru₂(OAc)₄]NO₃ | M Mukaida, T Nomura & T |
| | | Ishimori, Bull. Chem. Soc. |
| | | Japan, (1972), 45, 2143-7 |
| 12 | [Ru₂(OCOEt)₄]NO₃ | A Bino, F A Cotton & T R |
| | | Felthouse, Inorg. Chem. |
| | | (1979), 18, 2599-2604 |
| 13 | K₃[Ru(ox)₃] | C M Che, S S Kwong, C K |
| | | Poon, T F Lai & T C W |
| | | Mak Inorg. Chem. (1985), |
| | | 24, 1359-63 |
| 14 | [Ru₂(OAc)₄]Cl | R W Mitchell, A Spencer & |
| | | G Wilkinson |
| | | J. Chem. Soc. Dalton |
| | | Trans., (1973), 846-54 |
| 15 | [Ru(NH₃)₅Cl]Cl₂ | A D Allen, F Bottomley, |
| | | R O Harris, V P Reinsalu & |
| | | C V Senoff |
| | | J. Amer. Chem. Soc. |
| | | (1967), 89, 5595-5599 |
| 16 | [Ru(en)₃]I₃ | T J Meyer & H Taube |
| | | Inorg. Chem. (1968), 7, |
| | | 2369-2379 |
| 17 | K[RuCl₄(phen)]H₂O | B R James & R S |
| | | McMillan |
| | | Inorg. Nucl. Chem. Lett. |
| | | (1975), 11 (12) 837-9 |
| 18 | [Ru(cyclam)Cl₂]Cl | P K Chan, D A Isabirye & |
| | | C K Poon |
| | | Inorg. Chem. (1975), 14, |
| | | 2579-80 |
| 19 | K[RuCl₄(bipy)] | B R James & R S |
| | | McMillan |
| | | Inorg. Nucl. Chem. Lett. |
| | | (1975), 11 (12), 837-9 |
| 20 | [RuCl₃(dmso)₂(NH₃)] | Patent: International |
| | | Publication No |
| | | WO 91/13553 |
| 21 | [Ru(NH₃)₆]Cl₃ | Matthey Catalogue Sales: |
| | | Cat No [190245] |
| 22 | cis-[RuCl₂(dmso)₄] | E A Alessio, G Mestroni, |
| | | G Nardin, W M Attia, |
| | | M Calligaris, G Sava & |
| | | S Zorget |
| | | Inorg. Chem. (1988), 27, |
| | | 4099-4106 |
| 23 | cis-[RuCl₂(dmso)₃(NH₃)] | M Henn, E Alessio, |
| | | G Mestroni, M Calligaris & |
| | | W M Attia |
| | | Inorg. Chim, Acta, (1991), |
| | | 187, 39-50 |
| 24 | [RuCl₃(dmso)₃] | E Alessio, G Balducci, |
| | | M Calligaris, G Costa, |
| | | W M Attia & G Mestroni |
| | | Inorg. Chem. (1991), 30, |
| | | 609-618 |
| 25 | [Ru(mtc)₃] | A R Hendrickson, J M |
| | | Hope R L Martin |
| | | J. Chem. Soc. Dalton |
| | | Trans. (1976), 20, 2032-9 |
| 26 | [Ru(maltol)₃] | W P Griffith & S J Greaves |
| | | Polyhedron, (1988), 7 (19), |
| | | 1973-9 |
| 27 | [Ru(acac)₂MeCN)₂]CF₃SO₃ | Y Kasahara, T Hoshino |
| | | K. Shimizu & G P Sato |
| | | Chem. Lett. (1990), 3, |
| | | 381-4 |
| 28 | K₂[RuCl₅(H₂O)] | Matthey Catalogue Sales: |
| | | Cat No [190094] |
| 29 | [Os(ox)(bipy)₂].H₂O | D A Buckingham, F P |
| | | Dwyer, H A Goodwin & |
| | | A M Sargeson Aust. J. |
| | | Chem. (1964), 325-336 |
| | | G M Bryant, J E Fergusson |
| | | & H K J Powell |
| | | Aust. J. Chem. (1971), |
| | | 24 (2), 257-73 |
| 30 | [Ru(NH₃)₄Cl₂]Cl | S D Pell, M M Sherban, |
| | | V Tramintano & M J |
| | | Clarke |
| | | Inorg Synth, (1989), 26, |
| | | 65. |
| 31 | [Ru(Hedtra)(dppm)] | M M Taqui Khan, |
| | | K Venkatasubramanian, |
| | | Z Shirin, M M Bhadbhade |
| | | J Chem Soc Dalt Trans |
| | | (1992), 885-890 |
| 32 | Ru(oep)Ph | M Ke, S J Rettig, B R |
| | | James and D Dolphin |
| | | J Chem Soc Chem |
| | | Commun (1987), 1110 |

A number of new compounds were prepared according to the following protocols. The first four compounds are examples of ruthenium complexes of formula [Ru(H₀₋₆L^{II})₁₋₃Y _{O-2}Cl₀₋₄]^{(0-4)±} (Formula II), the subsequent two are examples of [M₁₋₃Y₁₋₈Cl₀₋₁₈]^{(0-6)±} (Formula III).

Preparation of [Ru(Hhedtra)acac].H₂O

Excess acetylacetone (1 cm³) was added to an aqueous solution (5 cm³) of K[Ru(hedtra)Cl] (0.5 g). The solution colour changed to violet. The mixture was warmed for 20 minutes then left to stand at room temperature for 20 minutes. The violet solution was extracted with chloroform (20 cm³). The extraction was repeated twice more. A violet product precipitated from the aqueous fraction. The product was filtered, washed in acetone and dried in vacuo, yield 0.1 g (18%).

AnaL Calc. for C₁₅H₂₅O_{1O}N₂Ru: C, 36.43; H, 5.11; N, 5.70. Found: C, 36.16; H, 5.42; N, 5.61%.

Preparation of [Ru(Hhedtra)trop]2H₂O.

A three-fold excess of tropolone (0.78 g) dissolved in 50:50 water/absolute ethanol (5 cm³) was added to a warm aqueous solution of K[Ru(hedtra)Cl] (10 cm³). The mixture was heated for 1 hour. On cooling, the dark green mixture was extracted with 3×2O cm³ portions of dichloromethane. On standing, a dark green product precipitated from the aqueous fraction. The product was filtered, washed with water (1 cm²), either and dried in vacuo, yield 0.4 g (36%).

Anal. Calc. for C₁₇H₂₂N₂O₉Ru.2H₂O: C,38.13; H, 4.86; N 5.23. Found C 38.55; H, 4.67; N 5.28%.

Preparation of [Ru(H₃dtpa)Cl]

K₂[RuCI₅H₂O].×H₂O (1 g) was suspended in HClO₄ (15 cm³, 1 mM) and diethylenetrianiinepentaacetic acid (1.05 g) added. The reaction mixture was heated under reflux for 1.5 hours forming a yellow/brown solution. On cooling a yellow product crystallised which was collected by filtration, washed with 90% absolute ethanol/water, diethyl ether and dried in vacuo, yield 0.75 g, 53%.

Anal. calcd. for C₁₄H₂₁N₃0₁₀ClRu: C, 31.85; H, 3.98; N, 7.96; Cl, 6.73. Found: C, 29.77; H, 3.81; N, 7.36; Cl, 6.64.

Preparation of K[RuHHBEDCl]3H₂O

0.41 g of K₂[RuCl₅]×H₂O was dissolved in water (20 ml). To this solution was added 1 equivalent (0.39 g) of N,N′di (2-hydroxy-benzyl)ethylene-diamine N,N-diacetic acid (hbed) dissolved in water (50 ml) with KOH (0.12 g) and MEOH (1 ml.). This mixture was heated at reflux for 90 minutes. Upon cooling a dark, insoluble precipitate formed. This material was removed by filtration and the resulting red-violet solution was taken to dryness by rotary vaporation. Trituration with water and washing with acetone yielded 90 mg of a dark solid.

AnaL calcd. for C₁₈H₂₂N₂O₉RuClK: C, 36.89; H, 3.96; N, 4.78; Cl, 6.04. Found: C, 37.09; H, 4.23; N, 4.92; Cl, 6.28.

Preparation of Ru(S₂CNCH₂CH₂NMeCH₂CH₂),½H₂O

Me₄N[S₂CNCH₂CH₂NMeCH₂CH₂] was made by the same standard method and crystallised from methanol-ether in 71% yield.

RuCl₃×H₂O, 0.50 g, 2.15 mmol was refluxed in 30 ml of methanol for 10 minutes and cooled. 1.87 g, 7.50 mmol of Me₄N[S₂CNCH₂CH₂NMeCH₂CH₂] was added and the mixture refluxed for 16 hours. After cooling 0.72 g of crude product was filtered off, dissolved in dichloromethane and filtered. The filtrate was loaded into 15 cc of basic alumina and eluted with dichloromethane. Removal of solvent and crystallisation from dichloromethane with ether by vapour-phase diffusion gave 0.51 g 0.80 mmol, 37% of brown-black crystals, Ru(S₂CNCH₂CH₂NMeCH₂CH₂)₃½H₂O.

Analysis for C₁₈H₃₄N₆O₅RuS₆: Calc: C, 34.00; H, 5.39; N, 13.22; S, 30.25. Found: C, 34.21; H, 5.47; N, 13.12; S, 30.36.

Preparation of Ru[S₂P(OC₂H₂OC₂H₄OMe)₂]₃

Ru[S₂P(OC₂H₂OC₂H₄OMe)₂]₃ was made by standard method and crystallised from methanol in 76% yield.

RuCl₃×H₂O, 1.00 g, 4.30 mmol was refluxed in 50 ml of 0.1 N HCl with 1 ml of ethanol for 20 minutes and cooled. To this solution was added 5.28 g (excess) K[S₂P(OC₂H₄OC₂H₄OME)₂] and the mixture stirred at 30′ C. for 1 hour. The reaction mixture was extracted with dichloromethane and the solvent removed. The residue was extracted with ether-hexane and solvents removed. This residue was crystallised from 25 ml of hot ether by cooling to −20° C. giving 2.98 of red crystals. 2.41 g of the crude product was purified by chromatography on 60 cc of silica gel with 5% ethanol in ether. The first band was collected, reduced to dryness and crystallised from ether by cooling to −20° C . The yield of red crystals, Ru(S₂P{OC₂H₄OC₂OMe}₂)₃, was 2.16 g, 56%.

Analysis for C₃₀H₆₆O₁₈P₃RuS₆: Calc: C, 32.72; H, 6.04; S, 17.47. Found: C, 32.68; H, 6.08; S, 17.16.

In the in vitro tests, which were carried out in an atmosphere of argon, each compound (1×1O⁴ moles) was dissolved in double-distilled deionized and deoxygenated water. The resulting solution was placed in a three-necked pear-shaped flask and stirred by a magnetic stirrer at constant speed of 1000 rpm, at a constant temperature in the range 20° C.-24° C. A manometer was attached to the flask, purified, dried nitric oxide gas (known volume in the range 3-5 cm³) was introduced via a septum, using a gas syringe, at atmospheric pressure into the headspace above the reaction solution. The pressure within the flask was recorded periodically over a period of one hour.

A control experiment was carried out according to the above but without any complex present. The recorded pressures in association with the results of the control experiment were analyzed in order to determine the rate of NO uptake as a function of time for e each compound tested.

On completion of each in vitro test, the reaction solution was freeze-dried. An infrared spectrum of the freeze-dried product provided information on metal-NO bond formation.

In the in vitro cell culture tests, murine (RAW264) macrophage cell lines, which can be induced to produce nitric oxide, were seeded, 10⁶ cells/well, onto 24 well culture plates of 2 ml volume per well, in Eagles modified minimal essential medium (MEM) plus 10% foetal bovine serum without phenol red.

The cells were activated to produce nitric oxide, with 10, μg/ml lipopolysaccharide and 100 units/ml interferony γ for 18 hours. Concurrently, test compounds made up in MEM were added at non-cytotoxic concentrations.

Control cells as above, which were activated to produce nitric oxide as above, but to which no test compound was added, were used as a measure of the amount of nitric oxide produced by the cells during the tests.

Background nitric oxide was assessed by measurement of nitrate and nitrite in cells which were not activated.

Cell viability was confirmed by Trypan blue dye exclusion at the end of the incubation period.

Nitric oxide was determined by measurement of nitrate and nitrite in the cell supernatant. These anions are the stable end-products of reactions of NO in solution. Such reactions may or may not be catalysed in biological systems. The sum of nitrite and nitrate concentrations gives the total NO production. Nitrite was determined using the Griess reaction in which nitrite reacts with 1% sulphanilamide in 5% H₃PO₄/O.1% naphthylethylenedianine dihydrochloride to form a chromophore absorbing at 540 nm. Nitrate was determined by reducing nitrate to nitrite with a bacterial nitrate reductase from Pseudomonas oleovorans and then measuring nitrite with the Griess reaction. In the absence of test compounds nitrite concentration plus nitrate concentration is equal to total nitric oxide production. The effect of test compounds on available nitric oxide (measured as nitrite+nitrate) was determined. The reduction in available nitric oxide compared with the control level may be taken as an indication of the degree of binding of NO by the test compounds.

In the ex vivo tests, segments of rat tail artery (0.8-1.5 cm) were dissected free from normotensive adult Wistar rats. The arteries were internally perfused with Krebs solution (mM: NaCl 118, KCl 4.7, NaHCO₃ 25, NaH₂PO₄ 1.15, CaCl₂ 2.5, MgCl₂ 1.1, glucose 5.6 and gassed with 95% O₂/5% CO₂ to maintain a pH of 7.4) in a constant flow perfusion apparatus. A differential pressure transducer located upstream of the vessel detected changes in back pressure. The rat tail artery preparation was pre-contracted with 6.5 μM phenylephrine to give a physiologically normal pressure of 100-120 mm Hg. The pre-contracted vessels were then perfused with the test compound. The arteries were perfused with Krebs solution between applications of test compound to wash out the test compound.

Pressure changes in the system served to indicate artery vasoconstriction. The vasoconstriction is a direct result of the removal of endogenous nitric oxide (edrf) from the endothelial cells of the rat tail artery.

Results

The results of the in vitro, in vitro cell culture and ex-vivo tests were as follows:

In vitro Tests

### Example I

### K[Ru(Hedta)Cl]₂H₂O

A pressure decrease indicated binding of NO to the metal compound.

The IR spectrum showed a peak at 1897 cm⁻¹, indicating the presence of a RU—NO bond.

### Example 2

### [Ru(H2edta)(acac)]

The IR spectrum showed a peak at 1896 cm⁻¹, indicating the presence of a RU—NO bond.

### Example 3

### K[Ru(hedtra)Cl]H₂O

A pressure decrease indicated binding of NO to the metal compound.

The IR spectrum showed a peak at 1889 cm⁻¹, indicating the presence of a RU—NO bond.

### Example 4

### K[Ru(dipic)₂]H₂O

A pressure decrease indicated binding of NO to the metal compound.

The IR spectrum showed a peak at 1915 cm₋₁, indicating the presence of a RU—NO bond.

### Example 5

### (H₂pic)[RuCl₂(pic)₂](Hpic)H₂O

The IR spectrum showed a peak at 1888 cm⁻¹, indicating the presence of a RU—NO bond.

### Example 6

### K[Ru(H₂edta)Cl₂]H₂O

A pressure decrease indicated binding of NO to the metal compound.

The IR spectrum showed a peak at 1896 cm⁻¹, indicating the presence of a RU—NO bond.

### Example 7

### K[Ru(Hnta)₂]½H₂O

A pressure decrease indicated binding of NO to the metal compound.

The IR spectrum showed a peak at 1889 cm⁻¹, indicating the presence of a RU—NO bond.

### Example 8

### K[Ru(H₂dtpa)Cl]H₂O

A pressure decrease indicated binding of NO to the metal compound.

The IR spectrum showed a peak at 1905 cm⁻¹, indicating the presence of a RU—NO bond.

### Example 9

### [Ru₃O(lac)₆](lac)

The IR spectrum showed a peak at 1884 cm⁻¹, indicating the presence of a RU—NO bond.

### Example 10

### [RU₃O(OAc)₆](OAc)

The IR spectrum showed a peak at 1877 cm⁻¹, indicating the presence of a RU—NO bond.

### Example 11

### [RU₂(OAc)₄]NO₃

The IR spectrum showed a peak at 1891 cm⁻¹, indicating the presence of a RU—NO bond.

### Example 12

### [Ru(OCOEt)₄]NO₃

The IR spectrum showed a peak at 1891 cm⁻¹, indicating the presence of a RU—NO bond.

### Example 13

### K₃[R-(ox)₃]

The IR spectrum showed a peak at 1889 cm⁻¹, indicating the presence of a RU—NO bond.

### Example 14

### [Ru₂(OAc)₄]Cl

The IR spectrum showed a peak at 1895 cm⁻¹, indicating the presence of a RU—NO bond.

### Example 15

### [Ru(NH₃)₅Cl]Cl₂

The IR spectrum showed two peaks at 1909 cm⁻¹ and 1928 cm⁻¹, indicating the presence of a RU—NO bond.

### Example 16

### [Ru(en)₃]I₃

The IR spectrum showed a peak at 1906 cm⁻¹, indicating the presence of a RU—NO bond.

### Example 17

### K[RuCl₄(phen)]H₂O

The IR spectrum showed a peak at 1904 cm⁻¹, indicating the presence of a RU—NO bond.

### Example 18

### [Ru(cyclam)Cl₂]Cl

The IR spectrum showed a peak at 1895 cm⁻¹, indicating the presence of a RU—NO bond.

### Example 19

### K[RuCl₄(bipy)]

The IR spectrum showed a peak at 1885 cm⁻¹, indicating the presence of a RU—NO bond.

### Example 20

### [RuCl₃(dmso)₂(NH₃)]

The IR spectrum showed a peak at 1889 cm⁻¹, indicating the presence of a RU—NO bond.

### Example 21

### [Ru(NH₃)₆]Cl₃

The IR spectrum showed a peak at 1910 cm−1, indicating the presence of a RU—NO bond.

### Example 22

### cis-[RuCl₂(dmso)₄]

The IR spectrum showed a peak at 1881 cm⁻¹, indicating the presence of a RU—NO bond.

### Example 23

### cis-[RuCl₂(dmso)₃(NH₃)]

The IR spectrum showed a peak at 1893 cm⁻¹, indicating the presence of a RU—NO bond.

### Example 24

### [RuCl₃(dMso)₃]

The IR spectrum showed a peak at 1880 cm⁻¹, indicating the presence of a RU—NO bond.

### Example 25

### [Ru(mtc)₃]

The IR spectrum showed a peak at 1862 cm⁻¹, indicating the presence of a RU—NO bond.

### Example 26

### [Ru(maltol)₃]

The IR spectrum showed a peak at 1866 cm⁻¹, indicating the presence of a RU—NO bond.

### Example 27

### [Ru(acac)₂(MeCN)₂]CF₃SO3

The IR spectrum showed a peak at 1899 cm⁻¹, indicating the presence of a RU—NO bond.

### Example 28

### K₂[RuCl₅(H₂O)]

The IR spectrum showed a peak at 1903 cm⁻¹, indicating the presence of a RU—NO bond.

### Example 29

### [Os(ox)(bipy)₂]H₂O

The IR spectrum showed a peak at 1894 cm⁻¹, indicating the presence of a Os—NO bond.

In vitro Cell Culture Tests

Results are shown in Table 4.

### Example 1

### K[Ru(Hedta)Cl]2H₂O

Available nitric oxide was reduced in a dose-dependent fashion with a maximum reduction of 75% at a concentration of 100 μM.

### Example 2

### [Ru(H₂edta)(acac)]

Available nitric oxide was reduced by 82% at 100 μM test compound.

### Example 3

### K[Ru(Hedtra)Cl]H₂O

Available nitric oxide was reduced by 42% at 100 μM.

### Example 6

### K[Ru(H₂edta)Cl₂]H₂O

Available nitric oxide was reduced by 77% at 100 μM test compound.

### Example 14

### [RU₂(OAc)₄]Cl

Available nitric oxide was reduced by 47% at 100 μM.

### Example 15

### [Ru(NH₃)₅Cl]Cl₂

Available nitric oxide was reduced by 86% at 100 μM test compound.

### Example 26

### [Ru(maltolato)₃]

Available nitric oxide was reduced by 71% at 100 μM.

TABLE 4

| | | | % Decrease of Available |
|---|---|---|---|
| | | | Nitric Oxide |
| Example 1 | 27 | μM | 12 |
| | 50 | μM | 23 |
| | 100 | μM | 75 |
| Example 2 | 100 | μM | 82 |
| Example 3 | 100 | μM | 42 |
| Example 6 | 100 | μM | 77 |
| Example 14 | 100 | μM | 47 |
| Example 15 | 100 | μM | 86 |
| Example 26 | 100 | μM | 71 |

Ex-vivo Tests

### Example 2

Application of test compound resulted in a dose-dependent vasoconstriction at 10 μM and 100 μM. This effect was reversible by washout with Krebs solution.

### Example 3

Application of test compound resulted in a dose-dependent vasoconstriction at 10 μM and 100 μM. This effect was reversible by washout with Krebs solution.

### Example 14

Application of test compound resulted in a dose-dependent vasoconstriction at 10 μM and 100 μM. This effect was reversible by washout with Krebs solution.

### Example 15

Application of test compound resulted in a dose-dependent vasoconstriction at 10 μM and 100 μM. This effect was reversible by washout with Krebs solution.

### Example 26

Application of test compound resulted in a dose-dependent vasoconstriction at 10 μM and 100 μM and 1000 μM. This effect was reversible by washout with Krebs solution.

TABLE 5

| | | | % Vasoconstriction |
|---|---|---|---|
| Example 2 | 10 | μM | 20 |
| | 100 | μM | 69 |
| Example 3 | 10 | μM | 17 |
| | 100 | μM | 59 |
| Example 14 | 10 | μM | 11 |
| | 100 | μM | 40 |
| Example 15 | 10 | μM | 16 |
| | 100 | μM | 86 |
| Example 26 | 10 | μM | 10 |
| | 100 | μM | 18 |
| | 1000 | μM | 25 |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated by reference for all purposes.

Citation of the above documents is not intended as an admission that any of the foregoing is pertinent prior art, nor does it constitute any admission as to the contents or date of these documents.

It is claimed:

1. A method of modulating inflammation associated with trauma which comprises administering to a patient in need of such modulation an effective amount of a neutral, anionic, or cationic metal complex of Formula I
[Mₐ(X_{b}L)_{c}Y_{d}Zₑ]ⁿ^{_{±}} Formula I,
where:
M is a ruthenium ion;
X is cation or a mixture of cations;
L is a ligand, or mixture of ligands each containing at least two different donor atoms selected from nitrogen, oxygen and sulphur donor atoms;
Y is a ligand, or a mixture of the same or different ligands each containing at least one donor atom, which donor atom is selected from nitrogen, oxygen, sulphur, carbon and phosphorus, provided that the ligand is not a sulphoxide group, 1,10-phenanthroline or substituted 1,10-phenanthroline, substituted 2,2-bypyridine or dihydrophenazine; and
Z is a halide or pseudohalide ion or a mixture of halide ions and pseudohalide ions;
a=1-3; b=0-12; c=0-18; d=0-18; e=0-18; and n=1-10;
provided that at least one of c, d and e is 1 or more;
and where c is 0; b is also 0;
and where a is 1; c, d and e are not greater than 9 in total;
and where a is 3; c, d and e are not greater than 18 in total;
and where d is 3, Y is not a basic heterocyclic compound containing at least one nitrogen atom.

2. The method of claim 1 wherein the trauma is associated with cardiac bypass surgery, cardiac valve surgery, chronic congestive heart failure, heart transplantation, ischemic reperfusion, or angioplasty.

3. The method of claim 1 wherein the trauma is caused by surgery.

4. The method of claim 1 wherein the modulation reduces tissue damage in the patient.

5. The method of claim 4 wherein the modulation causes a protective effect against the impairment of an organ function.

6. The method of claim 5 wherein the organ is selected from the group consisting of brain, liver, kidney, lung or cardiac muscle.

7. The method of claim 1 wherein the ruthenium ion is in oxidation state III.

8. The method of claim 1 wherein X is a mono-, di-, or tri- valent cation.

9. The method of claim 8 wherein X is H⁺, K⁺, Na⁺, NH₄⁺, or Ca²⁺.

10. The method of claim 1 wherein L is a polydentate aminocarboxylate ligand.

11. The method of claim 1 wherein L is ethylenediamine-N,N′-diacetic acid (edda), ethylenediaminetetraacetic acid (edta), nitrilotriacetic acid (nta), dipicolinic acid (dipic), picolinic acid (pic), diethylenetriaminepentaacetic acid (dtpa), thiobis (ethylenenitrilo) tetraacetic acid (tedta), dithioethanebis (ethylenenitrilo) tetraacetic acid (dtedta) or N-(2-hydroxyethyl) ethylenediamine-triacetic acid (hedtra).

12. The method of claim 1 wherein the donor atom is nitrogen present as ammine, amine, amide, nitrile or nitride, or derivatives thereof.

13. The method of claim 1 wherein said donor atom is oxygen present as carboxylic acid, ester, water, oxide, hydroxide, acetate, lactate, propionate, oxalate, or maltolate.

14. The method of claim 1 wherein the donor atom is sulfur present as sulfoxide, dialkylsulphide, dithiocarbamate, or dithiophosphate.

15. The method of claim 1 wherein the donor atom is carbon present as carbon monoxide or isocyanide.

16. The method of claim 1 wherein the donor atom is phosphorus present as trialkylphosphine.

17. The method of claim 1 wherein Z is a halide.

18. The method of claim 17 wherein Z is chloride.

19. The method of claim 1 wherein the complex is an optionally hydrated ruthenium complex of Formula II
[Ru(H₀₋₆L^{II})₁₋₃Y₀₋₂Cl₀₋₄]^{(0-4)±} Formula II,
where L^{II} is an amide or ester or derivative thereof, or a polydentate aminocarboxylate ligand selected from edda, edta, nta, dipic, pic, dtpa, hedtra, tedta or dtedta or a mixture of these, and Y is selected from the group consisting of acetylacetone (acac), a β-diketonate, water, carboxylate, bidentate carboxylate, catechol, kojic acid, maltol, hydroxide, tropolone, malonic acid, oxalic acid, 2,3-dihydroxynaphthalene, squaric acid, acetate, a sulphate and a glycolate.

20. The method of claim 1 wherein the complex is an optionally hydrated complex of Formula III
[M₁₋₃Y₁₋₁₈Cl₀₋₁₈]^{(0-6)±} Formula III,
wherein M is a ruthenium ion and Y is a sulphur donor ligand other than a sulphoxide group and a pharmaceutically acceptable carrier thereof.

21. The method of claim 1 wherein the complex is an optionally hydrated complex of Formula IIIa
[M^{III}₁₋₃Y^{III}₁₋₁₈Cl₀₋₁₈]^{(0-6)±} Formula IIIa,
where M^{III} is ruthenium and Y^{III} is an oxygen-donor ligand selected from the group consisting of acetate, lactate, water, oxide, propionate, oxalate, maltolate and a combination of these.

22. The method of claim 1 wherein the complex is an optionally hydrated complex of Formula IV
[RuY^{IV}₁₋₉Cl₁₋₉]^{(0-4)±} Formula IV
where Y^{IV} is a nitrogen-donor ligand selected from the group consisting of ammine, ethylenediamine (en), pyridine (py), 1,4,8,11-tetraazacyclotetradecane (cyclam), 2,3,7,8,12,13,17,18-oxtaethyl-prophyrin (oep) and a combination of these, provided that when the complex is of formula [RuY^{IV}₃Cl₁₋₉]^{(0-4)±}, Y is not a basic heterocyclic compound containing at least one nitrogen atom.

23. The method of claim 1 wherein the complex is an optionally hydrated complex of Formula V
[M₁₋₃Y^{V}₁₋₁₈Cl₀₋₁₈]^{(0-6)±} Formula V
wherein Y^{V} is a combination of donor ligands selected from the group consisting of ammine, dimethylsulphoxide (dmso), oxalate, 2,2-bipyridine (bipy), acetylacetone (acac), and acetonitrile (MeCN).

24. A method of modulating matrix metalloproteinase activity which comprises administering to a patient in need of such modulation an effective amount of a neutral, anionic, or cationic metal complex of the Formula I
[Mₐ(X_{b}L)_{c}Y_{d}Zₑ]ⁿ^{_{±}} Formula I,
where:
M is a ruthenium ion;
X is cation or a mixture of cations;
L is a ligand, or mixture of ligands each containing at least two different donor atoms selected from nitrogen, oxygen and sulphur donor atoms;
Y is a ligand, or a mixture of the same or different ligands each containing at least one donor atom, which donor atom is selected from nitrogen, oxygen, sulphur, carbon, and phosphorus, provided that the ligand is not a sulphoxide group, 1,10-phenanthroline or substituted 1,10-phenanthroline, substituted 2,2-bypyridine or dihydrophenazine; and
Z is a halide or pseudohalide ion or a mixture of halide ions and pseudohalide ions;
a=1-3; b=0-12; c=0-18; d=0-18; e=0-18; and n=1-10;
provided that at least one of c, d and e is 1 or more;
and where c is 0; b is also 0;
and where a is 1; c, d and e are not greater than 9 in total;
and where a is 3; c, d and e are not greater than 18 in total;
and where d is 3, Y is not a basic heterocyclic compound containing at least one nitrogen atom.

25. The method of claim 24 wherein the modulation ameliorates the side effects of inflammation.

26. The method of claim 25 wherein the inflammation is associated with rheumatoid arthritis, inflammatory bowel disease, systemic lupus erythematosis, acute transplantation/graft rejection, myasthenia gravis, progressive systemic sclerosis, multiple myeloma, atopic dermatitis, hyperimmunoglobin E, hepatitis B antigen, negative chronic active hepatitis, Hashimoto's thyroiditis, Familial Mediterranean fever, Grave's disease, autoimmune hemolytic anemia, or primary biliary cirrhosis.

27. The method of claim 24 wherein the matrix metalloproteinase activity is associated with tumor metastasis or tumor growth.

28. The method of claim 24 wherein the modulation reduces tissue damage in the patient.

29. The method of claim 24 wherein the modulation causes a protective effect against the impairment of an organ function.

30. The method of claim 29 wherein the organ is selected from the group consisting of brain, liver, kidney, lung, or cardiac muscle.

31. The method of claim 24 wherein the modulation results in a reduction in the dosage required for an accompanying vascular disease treatment or in a reduction in the amount of fluid required.

32. The method of claim 24 wherein the ruthenium ion is in oxidation state III.

33. The method of claim 24 wherein X is a mono-, di-, or tri- valent cation.

34. The method of claim 33 wherein X is H⁺, K⁺, Na⁺, NH₄⁺, or Ca²⁺.

35. The method of claim 24 wherein L is a polydentate aminocarboxylate ligand.

36. The method of claim 24 wherein L is ethylenediamine-N,N′-diacetic acid (edda), ethylenediaminetetraacetic acid (edta), nitrilotriacetic acid (nta), dipicolinic acid (dipic), picolinic acid (pic), diethylenetriaminepentaacetic acid (dtpa), thiobis (ethylenenitrilo) tetraacetic acid (tedta), dithioethanebis (ethylenenitrilo) tetraacetic acid (dtedta) or N-(2-hydroxyethyl) ethylenediamine-triacetic acid (hedtra).

37. The method of claim 24 wherein the donor atom is nitrogen present as ammine, amine, amide, nitrile or nitride, or derivatives thereof.

38. The method of claim 24 wherein said donor atom is oxygen present as carboxylic acid, ester, water, oxide, hydroxide, acetate, lactate, propionate, oxalate, or maltolate.

39. The method of claim 24 wherein the donor atom is sulfur present as sulfoxide, dialkylsulphide, dithiocarbamate, or dithiophosphate.

40. The method of claim 24 wherein the donor atom is carbon present as carbon monoxide or isocyanide.

41. The method of claim 24 wherein the donor atom is phosphorus present as trialkylphosphine.

42. The method of claim 24 wherein Z is a halide.

43. The method of claim 42 wherein Z is chloride.

44. The method of claim 24 wherein the complex is an optionally hydrated ruthenium complex of Formula II
[Ru(H₀₋₆L^{II})₁₋₃Y₀₋₂Cl₀₋₄]^{(0-4)±} Formula II,
where L^{II} is an amide or ester or derivative thereof, or a polydentate aminocarboxylate ligand selected from edda, edta, nta, dipic, pic, dtpa, hedtra, tedta or dtedta or a mixture of these, and Y is selected from the group consisting of acetylacetone (acac), a β-diketonate, water, carboxylate, bidentate carboxylate, catechol, kojic acid, maltol, hydroxide, tropolone, malonic acid, oxalic acid, 2,3-dihydroxynaphthalene, squaric acid, acetate, a sulphate and a glycolate.

45. The method of claim 24 wherein the complex is an optionally hydrated complex of Formula III
[M₁₋₃Y₁₋₁₈Cl₀₋₁₈]⁽⁰⁻⁶⁾^{_{±}} Formula III,
wherein M is a ruthenium ion and Y is a sulphur donor ligand other than a sulphoxide group and a pharmaceutically acceptable carrier thereof.

46. The method of claim 24 wherein the complex is an optionally hydrated complex of Formula IIIa
[M^{III}₁₋₃Y^{III}₁₋₁₈Cl₀₋₁₈]⁽⁰⁻⁶⁾^{_{±}} Formula IIIa,
where M^{III} is ruthenium and Y^{III} is an oxygen-donor ligand selected from the group consisting of acetate, lactate, water, oxide, propionate, oxalate, maltolate and a combination of these.

47. The method of claim 24 wherein the complex is an optionally hydrated complex of Formula IV
[RuY^{IV}₁₋₉Cl₁₋₉]^{(0-4)±} Formula IV
where Y^{IV} is a nitrogen-donor ligand selected from the group consisting of ammine, ethylenediamine (en), pyridine (py), 1,4,8,11 -tetraazacyclotetradecane (cyclam), 2,3,7,8,12,13,17,18-oxtaethyl-prophyrin (oep) and a combination of these, provided that when the complex is of formula [RuY^{IV}₃Cl₁₋₉]^{(0-4)±}, Y is not a basic heterocyclic compound containing at least one nitrogen atom.

48. The method of claim 24 wherein the complex is an optionally hydrated complex of Formula V
[M₁₋₃Y^{V}₁₋₁₈Cl₀₋₁₈]^{(0-6)±} Formula V
wherein Y^{V} is a combination of donor ligands selected from the group consisting of ammine, dimethylsulphoxide (dmso), oxalate, 2,2-bipyridine (bipy), acetylacetone (acac), and acetonitrile (MeCN).

49. A method of improving vascular or hemodynamic function associated with trauma which comprises administering to a patient in need of such improvement an effective amount of a neutral, anionic, or cationic metal complex of Formula I
[Mₐ(X_{b}L)_{c}Y_{d}Zₑ]ⁿ^{_{±}} Formula I,
where:
M is a ruthenium ion;
X is cation or a mixture of cations;
L is a ligand, or mixture of ligands each containing at least two different donor atoms selected from nitrogen, oxygen and sulphur donor atoms;
Y is a ligand, or a mixture of the same or different ligands each containing at least one donor atom, which donor atom is selected from nitrogen, oxygen, sulphur, carbon, and phosphorus, provided that the ligand is not a sulphoxide group, 1,10-phenanthroline or substituted 1,10-phenanthroline, substituted 2,2-bypyridine or dihydrophenazine; and
Z is a halide or pseudohalide ion or a mixture of halide ions and pseudohalide ions;
a=1-3; b=0-12; c=0-18; d=0-18; e=0-18; and n=1-10;
provided that at least one of c, d and e is 1 or more;
and where c is 0; b is also 0;
and where a is 1; c, d and e are not greater than 9 in total;
and where a is 3; c, d and e are not greater than 18 in total;
and where d is 3, Y is not a basic heterocyclic compound containing at least one nitrogen atom.

50. The method of claim 49 wherein the ruthenium ion is in oxidation state III.

51. The method of claim 49 wherein X is a mono-, di-, or tri- valent cation.

52. The method of claim 51 wherein X is H⁺, K⁺, Na⁺, NH₄⁺, or Ca²⁺.

53. The method of claim 49 wherein L is a polydentate aminocarboxylate ligand.

54. The method of claim 49 wherein L is ethylenediamine-N,N′-diacetic acid (edda), ethylenediaminetetraacetic acid (edta), nitrilotriacetic acid (nta), dipicolinic acid (dipic), picolinic acid (pic), diethylenetriaminepentaacetic acid (dtpa), thiobis (ethylenenitrilo) tetraacetic acid (tedta), dithioethanebis (ethylenenitrilo) tetraacetic acid (dtedta) or N-(2-hydroxyethyl) ethylenediamine-triacetic acid (hedtra)

55. The method of claim 49 wherein the donor atom is nitrogen present as ammine, amine, amide, nitrile or nitride, or derivatives thereof.

56. The method of claim 49 wherein said donor atom is oxygen present as carboxylic acid, ester, water, oxide, hydroxide, acetate, lactate, propionate, oxalate, or maltolate.

57. The method of claim 49 wherein the donor atom is sulfur present as sulfoxide, dialkylsulphide, dithiocarbamate, or dithiophosphate.

58. The method of claim 49 wherein the donor atom is carbon present as carbon monoxide or isocyanide.

59. The method of claim 49 wherein the donor atom is phosphorus present as trialkylphosphine.

60. The method of claim 49 wherein Z is a halide.

61. The method of claim 60 wherein Z is chloride.

62. The method of claim 49 wherein the complex is an optionally hydrated ruthenium complex of Formula II
[Ru(H₀₋₆L^{II}₁₋₃Y₀₋₂Cl₀₋₄]^{(0-4)±} Formula II,
where L^{II} is an amide or ester or derivative thereof, or a polydentate aminocarboxylate ligand selected from edda, edta, nta, dipic, pic, dtpa, hedtra, tedta or dtedta or a mixture of these, and Y is selected from the group consisting of acetylacetone (acac), a β-diketonate, water, carboxylate, bidentate carboxylate, catechol, kojic acid, maltol, hydroxide, tropolone, malonic acid, oxalic acid, 2,3-dihydroxynaphthalene, squaric acid, acetate, a sulphate and a glycolate.

63. The method of claim 49 wherein the complex is an optionally hydrated complex of Formula III
[M₁₋₃Y₁₋₁₈Cl₀₋₁₈]⁽⁰⁻⁶⁾^{_{±}} Formula III,
wherein M is a ruthenium ion and Y is a sulphur donor ligand other than a sulphoxide group and a pharmaceutically acceptable carrier thereof.

64. The method of claim 49 wherein the complex is an optionally hydrated complex of Formula IIIa
[M^{III}₁₋₃Y^{III}₁₋₁₈Cl₀₋₁₈]⁽⁰⁻⁶⁾^{_{±}} Formula IIIa,
where M^{III} is ruthenium and Y^{III} is an oxygen-donor ligand selected from the group consisting of acetate, lactate, water, oxide, propionate, oxalate, maltolate and a combination of these.

65. The method of claim 49 wherein the complex is an optionally hydrated complex of Formula IV
[RuY^{IV}₁₋₉Cl₁₋₉]^{(0-4)±} Formula IV
where Y^{IV} is a nitrogen-donor ligand selected from the group consisting of ammine, ethylenediamine (en), pyridine (py), 1,4,8,11-tetraazacyclotetradecane (cyclam), 2,3,7,8,12,13,17,18-oxtaethyl-prophyrin (oep) and a combination of these, provided that when the complex is of formula [RuY^{IV}₃Cl₁₋₉]^{(0-4)±}, Y is not a basic heterocyclic compound containing at least one nitrogen atom.

66. The method of claim 49 wherein the complex is an optionally hydrated complex of Formula V
[M₁₋₃Y^{V}₁₋₁₈Cl₀₋₁₈]^{(0-6)±} Formula V
wherein Y^{V} is a combination of donor ligands selected from the group consisting of ammine, dimethylsulphoxide (dmso), oxalate, 2,2-bipyridine (bipy), acetylacetone (acac), and acetonitrile (MeCN).
